(19) United States
(12) Patent Application Publication
(10) Pub. No.: US 2009/0151024 A1
(43) Pub. Date: Jun. 11, 2009

(54) Method for Controlling Rust Infections in Leguminous Plants

(76) Inventors: Jochen Dietz, Mannheim (DE); Reinhard Stierl, Freinsheim (DE); Siegfried Strathmann, Limburgerhof (DE)

Correspondence Address: BIRCH STEWART KOLASCH & BIRCH, PO BOX 747, FALLS CHURCH, VA, 22040-0747, US

(21) Appl. No.: 11/989,745

(22) PCT Filed: Jul. 31, 2006
(86) PCT No.: PCT/EP2006/064861

§ 371 Date: Jan. 30, 2008

(51) Int. Cl. A01H 5/00; A01N 43/40; A01N 43/58

(52) U.S. Cl. 800/298; 514/407; 514/354; 504/130; 504/139

(57) ABSTRACT

Method for controlling rust infections in leguminous plants by using heterocyclylcarboxanilides of the formula I

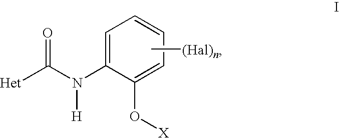

where n=0-4; X=C₁-C₄-haloalkyl;
Het=a pyrazole, thiazole or pyridine radical of the formula IIa, IIb or IIc

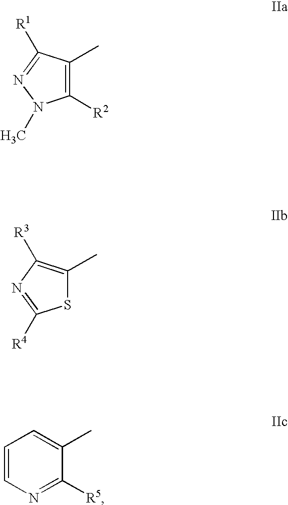

where
R¹ is C₁-C₄-alkyl or C₁-C₄-haloalkyl,
R² is H or halogen,
R³ is C₁-C₄-alkyl or C₁-C₄-haloalkyl,
R⁴ is C₁-C₄-alkyl or C₁-C₄-haloalkyl and
R⁵ is halogen, C₁-C₄-alkyl, C₁-C₄-haloalkyl, C₁-C₄-alkoxy, C₁-C₄-alkylthio, C₁-C₄-alkylsulfinyl or C₁-C₄-alkylsulfonyl,
mixtures of heterocyclylcarboxanilides of the formula I and a fungicidally active compound II from the group of the azoles, acylalanines, amine derivatives, anilinopyrimidines, dicarboximides, dithiocarbamates, heterocylic compounds, phenylpyrroles, cinnamides, and analogs, or other fungicides according to the description,
and also compositions and seed comprising these mixtures.

The invention relates to a method for controlling rust infections in leguminous plants.

Until recently, in the most important regions for the cultivation of leguminous plants (in particular soybeans) there were no infections with harmful fungi that were of economic significance.

However, over recent years, there has been an increase in severe rust infections of soybean crops in South America by the harmful fungi Phakopsora pachyrhizi and Phakopsora meibomiae. There have been considerable harvest and yield losses.

Most customary fungicides are unsuitable for controlling rust in soybeans, or their action against the rust fungus is unsatisfactory.

Surprisingly, it has now been found that heterocyclylcarboxanilides of the formula I

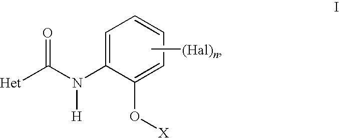

in which the variables are as defined below:
n is 0, 1, 2, 3 or 4;
Hal is halogen;
X is C₁-C₆-haloalkyl or C₂-C₆-haloalkenyl;
Het is a pyrazole, thiazole or pyridine radical of the formula IIa, IIb or IIc

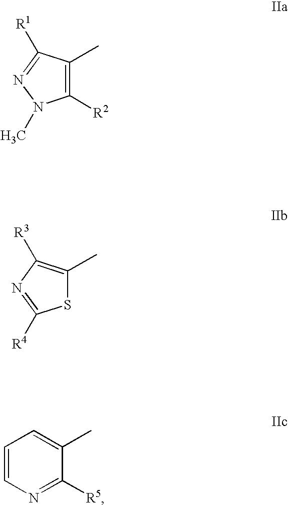

where
R¹ is C₁-C₄-alkyl or C₁-C₄-haloalkyl,
R² is hydrogen or halogen,
R³ is C₁-C₄-alkyl or C₁-C₄-haloalkyl,
R⁴ is C₁-C₄-alkyl or C₁-C₄-haloalkyl and
R⁵ is halogen, C₁-C₄-alkyl, C₁-C₄-haloalkyl, C₁-C₄-alkoxy, C₁-C₄-alkylthio, C₁-C₄-alkylsulfinyl or C₁-C₄-alkylsulfonyl,
   have excellent activity against rust fungus diseases of leguminous plants.

Before, carboxanilides of the type of the compounds I have been described as being particularly effective against botrytis (cf.; for example, EP-A 545 099 and EP-A 589 301).

The active compounds described in the present invention as mixing partners are generally known to the person skilled in the art (cf. http://www.hclrss.demon.co.uk/) and commercially available.

In formula I, halogen is fluorine, chlorine, bromine or iodine, preferably fluorine or chlorine;

C₁-C₄-alkyl is methyl, ethyl, n-propyl, 1-methylethyl, n-butyl, 1-methylpropyl, 2-methylpropyl or 1,1-dimethylethyl, preferably methyl or ethyl;
C₁-C₄-haloalkyl is a partially or fully halogenated C₁-C₄-alkyl radical, where the halogen atom(s) is/are in particular fluorine, chlorine and/or bromine, i.e., for example, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-bromo-2,2-difluoroethyl, 1,1,2,2-tetrafluoroethyl, 1,1,2-trifluoro-2-chloroethyl, 2-chloro-2-fluoroethyl, 2-chloro-2,2-difluoroethyl, 2,2-dichloro-2-fluoroethyl, 2,2,2-trichloroethyl, 1,1,2,2-tetrachloroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl, in particular halomethyl, with particular preference CH₂—Cl, CH(Cl)₂, CH₂—F, CH(F)₂, CF₃, CHFCl, CF₂Cl or CF(Cl)₂;
C₁-C₆-haloalkyl is a partially or fully halogenated C₁-C₆-alkyl radical, where the halogen atom(s) is/are in particular fluorine, chlorine and/or bromine, i.e., for example, a C₁-C₄-haloalkyl radical as mentioned above, or n-undecafluoropentyl or n-tridecafluorohexyl, in particular C₁-C₄-haloalkyl, with particular preference 2-bromo-2,2-difluoroethyl, 1,1,2,2-tetrafluoroethyl, 1,1,2-trifluoro-2-chloroethyl, 1,1,2,2-tetrachloroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl;
C₂-C₆-haloalkenyl is a partially or fully halogenated C₂-C₆-alkenyl radical, where the halogen atom(s) is/are in particular fluorine and/or chlorine, i.e., for example, 1-chlorovinyl, 2-chlorovinyl, 1,2-dichlorovinyl, 1,2,2-trichlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl, in particular 2-chloroallyl;
C₁-C₄-alkoxy is methoxy, ethoxy, n-propoxy, 1-methylethoxy, n-butoxy, 1-methylpropoxy, 2-methylpropoxy or 1,1-dimethylethoxy, preferably methoxy;
C₁-C₄-alkylthio is methylthio, ethylthio, n-propylthio, 1-methylethylthio, n-butylthio, 1-methylpropylthio, 2-methylpropylthio or 1,1-dimethylethylthio, preferably methylthio;
C₁-C₄-alkylsulfinyl is methylsulfinyl, ethylsulfinyl, n-propylsulfinyl, 1-methylethylsulfinyl, n-butylsulfinyl, 1-methylpropylsulfinyl, 2-methylpropylsulfinyl or 1,1-dimethylethylsulfinyl, preferably methylsulfinyl;
C₁-C₄-alkylsulfonyl is methylsulfonyl, ethylsulfonyl, n-propylsulfonyl, 1-methylethylsulfonyl, n-butylsulfonyl, 1-methylpropylsulfonyl, 2-methylpropylsulfonyl or 1,1-dimethylethylsulfonyl, preferably methylsulfonyl.

Particular preference is given to those compounds I in which the variables are as defined below, both on their own and in any combination with one another:
n is 0 or 1, in particular 0;
Hal is fluorine or chlorine;
X is C₁-C₆-haloalkyl, in particular C₁-C₄-haloalkyl, with particular preference 2-bromo-2,2-difluoroethyl, 1,1,2,2-tetrafluoroethyl, 1,1,2-trifluoro-2-chloroethyl, 1,1,2,2-tetrachloroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl, in particular 1,1,2,2-tetrafluoroethyl; Het is a pyrazole radical of the formula IIa

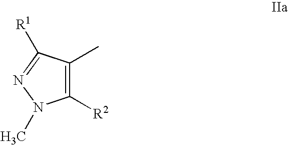

R¹ is methyl or halomethyl, in particular methyl, CHF₂ or CF₃, with particular preference CHF₂ or CF₃, in particular CHF₂;
R² is hydrogen, fluorine or chlorine, in particular hydrogen or fluorine, with particular preference hydrogen;
R³ is methyl or halomethyl, in particular methyl, CHF₂ or CF₃;
R⁴ is C₁-C₄-alkyl, in particular methyl;
R⁵ is halogen, methyl, halomethyl, methoxy, methylthio, methylsulfinyl or methylsulfonyl, in particular fluorine, chlorine, methyl, difluoromethyl, trifluoromethyl or methoxy.

Particular preference is also given to the heterocyclylcarboxanilides I listed in Tables A) to C) below, where n is in each case 0 and Het is a pyrazole radical IIa where R²=hydrogen.

TABLE A
Compounds I in which Het is a
pyrazole radical of the formula IIa:

| Com- | | | | |
|---|---|---|---|---|
| pound No. | R¹ | R² | X | Physical data |
| 1 | CH₃ | H | —CHF₂ | |
| 2 | CH₃ | H | —CF₃ | |
| 3 | CH₃ | H | —CH₂—CHF₂ | |
| 4 | CH₃ | H | —CH₂—CF₃ | |
| 5 | CH₃ | H | —CF₂—CHF₂ | 114-115° C. |
| 6 | CH₃ | H | —CF₂—CF₃ | |
| 7 | CH₃ | H | —CF₂—CHF—Cl | |
| 8 | CH₃ | H | —CF₂—CHF—CF₃ | |
| 9 | CH₃ | H | —CH(CF₃)₂ | |
| 10 | CH₃ | H | —CF₂—CF₂—CF₃ | |
| 11 | CH₃ | H | —CF(CF₃)₂ | |
| 12 | CH₃ | H | —CH₂—CF₂—Br | |
| 13 | CH₃ | H | —CH₂—CF₂—CHF₂ | |
| 14 | CH₃ | H | —CH₂—CF₂—CF₂—CF₃ | |
| 15 | CH₃ | H | —CF₂—CF₂—CF₂—CF₃ | |
| 16 | CH₃ | H | —CH₂—C(Cl)═CH₂ | |
| 17 | CH₂F | H | —CHF₂ | |
| 18 | CH₂F | H | —CF₃ | |
| 19 | CH₂F | H | —CH₂—CHF₂ | |
| 20 | CH₂F | H | —CH₂—CF₃ | |
| 21 | CH₂F | H | —CF₂—CHF₂ | 111-112° C. |
| 22 | CH₂F | H | —CF₂—CF₃ | |
| 23 | CH₂F | H | —CF₂—CHF—Cl | |
| 24 | CH₂F | H | —CF₂—CHF—CF₃ | |
| 25 | CH₂F | H | —CH(CF₃)₂ | |
| 26 | CH₂F | H | —CF₂—CF₂—CF₃ | |
| 27 | CH₂F | H | —CF(CF₃)₂ | |
| 28 | CH₂F | H | —CH₂—CF₂—Br | |
| 29 | CH₂F | H | —CH₂—CF₂—CHF₂ | |
| 30 | CH₂F | H | —CH₂—CF₂—CF₂—CF₃ | |
| 31 | CH₂F | H | —CF₂—CF₂—CF₂—CF₃ | |
| 32 | CH₂F | H | —CH₂—C(Cl)═CH₂ | |
| 33 | CHF₂ | H | —CHF₂ | |
| 34 | CHF₂ | H | —CF₃ | |
| 35 | CHF₂ | H | —CH₂—CHF₂ | |
| 36 | CHF₂ | H | —CH₂—CF₃ | |
| 37 | CHF₂ | H | —CF₂—CHF₂ | 117-119° C. |
| 38 | CHF₂ | H | —CF₂—CF₃ | |
| 39 | CHF₂ | H | —CF₂—CHF—Cl | 106-108° C. |
| 40 | CHF₂ | H | —CF₂—CHF—CF₃ | 97-99° C. |
| 41 | CHF₂ | H | —CH(CF₃)₂ | |
| 42 | CHF₂ | H | —CF₂—CF₂—CF₃ | |
| 43 | CHF₂ | H | —CF(CF₃)₂ | |
| 44 | CHF₂ | H | —CH₂—CF₂—Br | |
| 45 | CHF₂ | H | —CH₂—CF₂—CHF₂ | |
| 46 | CHF₂ | H | —CH₂—CF₂—CF₂—CF₃ | |
| 47 | CHF₂ | H | —CF₂—CF₂—CF₂—CF₃ | |
| 48 | CHF₂ | H | —CH₂—C(Cl)═CH₂ | |
| 49 | CF₃ | H | —CHF₂ | 88-89° C. |
| 50 | CF₃ | H | —CF₃ | 118-119° C. |
| 51 | CF₃ | H | —CH₂—CHF₂ | 122-124° C. |
| 52 | CF₃ | H | —CH₂—CF₃ | 85-86° C. |
| 53 | CF₃ | H | —CF₂—CHF₂ | 96-98° C. |
| 54 | CF₃ | H | —CF₂—CF₃ | |
| 55 | CF₃ | H | —CF₂—CHF—Cl | 94-95° C. |
| 56 | CF₃ | H | —CF₂—CHF—CF₃ | |
| 57 | CF₃ | H | —CH(CF₃)₂ | |
| 58 | CF₃ | H | —CF₂—CF₂—CF₃ | |
| 59 | CF₃ | H | —CF(CF₃)₂ | |
| 60 | CF₃ | H | —CH₂—CF₂—Br | |
| 61 | CF₃ | H | —CH₂—CF₂—CHF₂ | 101-102° C. |
| 62 | CF₃ | H | —CH₂—CF₂—CF₂—CF₃ | 93-95° C. |
| 63 | CF₃ | H | —CF₂—CF₂—CF₂—CF₃ | |
| 64 | CF₃ | H | —CH₂—C(Cl)═CH₂ | |
| 65 | CHF—Cl | H | —CHF₂ | |
| 66 | CHF—Cl | H | —CF₃ | |
| 67 | CHF—Cl | H | —CH₂—CHF₂ | |
| 68 | CHF—Cl | H | —CH₂—CF₃ | |
| 69 | CHF—Cl | H | —CF₂—CHF₂ | 111-112° C. |
| 70 | CHF—Cl | H | —CF₂—CF₃ | |
| 71 | CHF—Cl | H | —CF₂—CHF—Cl | |
| 72 | CHF—Cl | H | —CF₂—CHF—CF₃ | |
| 73 | CHF—Cl | H | —CH(CF₃)₂ | |
| 74 | CHF—Cl | H | —CF₂—CF₂—CF₃ | |
| 75 | CHF—Cl | H | —CF(CF₃)₂ | |
| 76 | CHF—Cl | H | —CH₂—CF₂—Br | |
| 77 | CHF—Cl | H | —CH₂—CF₂—CHF₂ | |
| 78 | CHF—Cl | H | —CH₂—CF₂—CF₂—CF₃ | |
| 79 | CHF—Cl | H | —CF₂—CF₂—CF₂—CF₃ | |
| 80 | CHF—Cl | | —CH₂—C(Cl)═CH₂ | |
| 81 | CF₂—Cl | H | —CHF₂ | |
| 82 | CF₂—Cl | H | —CF₃ | |
| 83 | CF₂—Cl | H | —CH₂—CHF₂ | |
| 84 | CF₂—Cl | H | —CH₂—CF₃ | |
| 85 | CF₂—Cl | H | —CF₂—CHF₂ | 100-102° C. |
| 86 | CF₂—Cl | H | —CF₂—CF₃ | |
| 87 | CF₂—Cl | H | —CF₂—CHF—Cl | |
| 88 | CF₂—Cl | H | —CF₂—CHF—CF₃ | |
| 89 | CF₂—Cl | H | —CH(CF₃)₂ | |
| 90 | CF₂—Cl | H | —CF₂—CF₂—CF₃ | |
| 91 | CF₂—Cl | H | —CF(CF₃)₂ | |
| 92 | CF₂—Cl | H | —CH₂—CF₂—Br | |
| 93 | CF₂—Cl | H | —CH₂—CF₂—CHF₂ | |
| 94 | CF₂—Cl | H | —CH₂—CF₂—CF₂—CF₃ | |
| 95 | CF₂—Cl | H | —CF₂—CF₂—CF₂—CF₃ | |
| 96 | CF₂—Cl | H | —CH₂—C(Cl)═CH₂ | |
| 97 | CF(Cl)₂ | H | —CHF₂ | |
| 98 | CF(Cl)₂ | H | —CF₃ | |
| 99 | CF(Cl)₂ | H | —CH₂—CHF₂ | |
| 100 | CF(Cl)₂ | H | —CH₂—CF₃ | |
| 101 | CF(Cl)₂ | H | —CF₂—CHF₂ | 118-119° C. |
| 102 | CF(Cl)₂ | H | —CF₂—CF₃ | |
| 103 | CF(Cl)₂ | H | —CF₂—CHF—Cl | |
| 104 | CF(Cl)₂ | H | —CF₂—CHF—CF₃ | |
| 105 | CF(Cl)₂ | H | —CH(CF₃)₂ | |
| 106 | CF(Cl)₂ | H | —CF₂—CF₂—CF₃ | |
| 107 | CF(Cl)₂ | H | —CF(CF₃)₂ | |
| 108 | CF(Cl)₂ | H | —CH₂—CF₂—Br | |
| 109 | CF(Cl)₂ | H | —CH₂—CF₂—CHF₂ | |
| 110 | CF(Cl)₂ | H | —CH₂—CF₂—CF₂—CF₃ | |
| 111 | CF(Cl)₂ | H | —CF₂—CF₂—CF₂—CF₃ | |
| 112 | CF(Cl)₂ | H | —CH₂—C(Cl)═CH₂ | |
| 113 | CHF—CH₃ | H | —CHF₂ | |
| 114 | CHF—CH₃ | H | —CF₃ | |
| 115 | CHF—CH₃ | H | —CH₂—CHF₂ | |
| 116 | CHF—CH₃ | H | —CH₂—CF₃ | |
| 117 | CHF—CH₃ | H | —CF₂—CHF₂ | |
| 118 | CHF—CH₃ | H | —CF₂—CF₃ | |
| 119 | CHF—CH₃ | H | —CF₂—CHF—Cl | |
| 120 | CHF—CH₃ | H | —CF₂—CHF—CF₃ | |
| 121 | CHF—CH₃ | H | —CH(CF₃)₂ | |
| 122 | CHF—CH₃ | H | —CF₂—CF₂—CF₃ | |
| 123 | CHF—CH₃ | H | —CF(CF₃)₂ | |
| 124 | CHF—CH₃ | H | —CH₂—CF₂—Br | |
| 125 | CHF—CH₃ | H | —CH₂—CF₂—CHF₂ | |
| 126 | CHF—CH₃ | H | —CH₂—CF₂—CF₂—CF₃ | |
| 127 | CHF—CH₃ | H | —CF₂—CF₂—CF₂—CF₃ | |
| 128 | CHF—CH₃ | H | —CH₂—C(Cl)═CH₂ | |
| 129 | CH₃ | F | —CHF₂ | |
| 130 | CH₃ | F | —CF₃ | |
| 131 | CH₃ | F | —CH₂—CHF₂ | |
| 132 | CH₃ | F | —CH₂—CF₃ | |
| 133 | CH₃ | F | —CF₂—CHF₂ | |
| 134 | CH₃ | F | —CF₂—CF₃ | |
| 135 | CH₃ | F | —CF₂—CHF—Cl | |
| 136 | CH₃ | F | —CF₂—CHF—CF₃ | |
| 137 | CH₃ | F | —CH(CF₃)₂ | |
| 138 | CH₃ | F | —CF₂—CF₂—CF₃ | |
| 139 | CH₃ | F | —CF(CF₃)₂ | |
| 140 | CH₃ | F | —CH₂—CF₂—Br | |
| 141 | CH₃ | F | —CH₂—CF₂—CHF₂ | |
| 142 | CH₃ | F | —CH₂—CF₂—CF₂—CF₃ | |
| 143 | CH₃ | F | —CF₂—CF₂—CF₂—CF₃ | |
| 144 | CH₃ | F | —CH₂—C(Cl)═CH₂ | |
| 145 | CH₂F | F | —CHF₂ | |
| 146 | CH₂F | F | —CF₃ | |
| 147 | CH₂F | F | —CH₂—CHF₂ | |
| 148 | CH₂F | F | —CH₂—CF₃ | |
| 149 | CH₂F | F | —CF₂—CHF₂ | |
| 150 | CH₂F | F | —CF₂—CF₃ | |
| 151 | CH₂F | F | —CF₂—CHF—Cl | |
| 152 | CH₂F | F | —CF₂—CHF—CF₃ | |
| 153 | CH₂F | F | —CH(CF₃)₂ | |
| 154 | CH₂F | F | —CF₂—CF₂—CF₃ | |
| 155 | CH₂F | F | —CF(CF₃)₂ | |
| 156 | CH₂F | F | —CH₂—CF₂—Br | |
| 157 | CH₂F | F | —CH₂—CF₂—CHF₂ | |
| 158 | CH₂F | F | —CH₂—CF₂—CF₂—CF₃ | |
| 159 | CH₂F | F | —CF₂—CF₂—CF₂—CF₃ | |
| 160 | CH₂F | F | —CH₂—C(Cl)═CH₂ | |
| 161 | CHF₂ | F | —CHF₂ | |
| 162 | CHF₂ | F | —CF₃ | |
| 163 | CHF₂ | F | —CH₂—CHF₂ | |
| 164 | CHF₂ | F | —CH₂—CF₃ | |
| 165 | CHF₂ | F | —CF₂—CHF₂ | |
| 166 | CHF₂ | F | —CF₂—CF₃ | |
| 167 | CHF₂ | F | —CF₂—CHF—Cl | |
| 168 | CHF₂ | F | —CF₂—CHF—CF₃ | |
| 169 | CHF₂ | F | —CH(CF₃)₂ | |
| 170 | CHF₂ | F | —CF₂—CF₂—CF₃ | |
| 171 | CHF₂ | F | —CF(CF₃)₂ | |
| 172 | CHF₂ | F | —CH₂—CF₂—Br | |
| 173 | CHF₂ | F | —CH₂—CF₂—CHF₂ | |
| 174 | CHF₂ | F | —CH₂—CF₂—CF₂—CF₃ | |
| 175 | CHF₂ | F | —CF₂—CF₂—CF₂—CF₃ | |
| 176 | CHF₂ | F | —CH₂—C(Cl)═CH₂ | |
| 177 | CF₃ | F | —CHF₂ | |
| 178 | CF₃ | F | —CF₃ | |
| 179 | CF₃ | F | —CH₂—CHF₂ | 100-102° C. |
| 180 | CF₃ | F | —CH₂—CF₃ | |
| 181 | CF₃ | F | —CF₂—CHF₂ | 91-93° C. |
| 182 | CF₃ | F | —CF₂—CF₃ | |
| 183 | CF₃ | F | —CF₂—CHF—Cl | |
| 184 | CF₃ | F | —CF₂—CHF—CF₃ | |
| 185 | CF₃ | F | —CH(CF₃)₂ | |
| 186 | CF₃ | F | —CF₂—CF₂—CF₃ | |
| 187 | CF₃ | F | —CF(CF₃)₂ | |
| 188 | CF₃ | F | —CH₂—CF₂—Br | |
| 189 | CF₃ | F | —CH₂—CF₂—CHF₂ | |
| 190 | CF₃ | F | —CH₂—CF₂—CF₂—CF₃ | |
| 181 | CF₃ | F | —CF₂—CF₂—CF₂—CF₃ | |
| 192 | CF₃ | F | —CH₂—C(Cl)═CH₂ | |
| 193 | CHF—Cl | F | —CHF₂ | |
| 194 | CHF—Cl | F | —CF₃ | |
| 195 | CHF—Cl | F | —CH₂—CHF₂ | |
| 196 | CHF—Cl | F | —CH₂—CF₃ | |
| 197 | CHF—Cl | F | —CF₂—CHF₂ | |
| 198 | CHF—Cl | F | —CF₂—CF₃ | |
| 199 | CHF—Cl | F | —CF₂—CHF—Cl | |
| 200 | CHF—Cl | F | —CF₂—CHF—CF₃ | |
| 201 | CHF—Cl | F | —CH(CF₃)₂ | |
| 202 | CHF—Cl | F | —CF₂—CF₂—CF₃ | |
| 203 | CHF—Cl | F | —CF(CF₃)₂ | |
| 204 | CHF—Cl | F | —CH₂—CF₂—Br | |
| 205 | CHF—Cl | F | —CH₂—CF₂—CHF₂ | |
| 206 | CHF—Cl | F | —CH₂—CF₂—CF₂—CF₃ | |
| 207 | CHF—Cl | F | —CF₂—CF₂—CF₂—CF₃ | |
| 208 | CHF—Cl | F | —CH₂—C(Cl)═CH₂ | |
| 209 | CF₂—Cl | F | —CHF₂ | |
| 210 | CF₂—Cl | F | —CF₃ | |
| 211 | CF₂—Cl | F | —CH₂—CHF₂ | |
| 212 | CF₂—Cl | F | —CH₂—CF₃ | |
| 213 | CF₂—Cl | F | —CF₂—CHF₂ | |
| 214 | CF₂—Cl | F | —CF₂—CF₃ | |
| 215 | CF₂—Cl | F | —CF₂—CHF—Cl | |
| 216 | CF₂—Cl | F | —CF₂—CHF—CF₃ | |
| 217 | CF₂—Cl | F | —CH(CF₃)₂ | |
| 218 | CF₂—Cl | F | —CF₂—CF₂—CF₃ | |
| 219 | CF₂—Cl | F | —CF(CF₃)₂ | |
| 220 | CF₂—Cl | F | —CH₂—CF₂—Br | |
| 221 | CF₂—Cl | F | —CH₂—CF₂—CHF₂ | |
| 222 | CF₂—Cl | F | —CH₂—CF₂—CF₂—CF₃ | |
| 223 | CF₂—Cl | F | —CF₂—CF₂—CF₂—CF₃ | |
| 224 | CF₂—Cl | F | —CH₂—C(Cl)═CH₂ | |
| 225 | CF(Cl)₂ | F | —CHF₂ | |
| 226 | CF(Cl)₂ | F | —CF₃ | |
| 227 | CF(Cl)₂ | F | —CH₂—CHF₂ | |
| 228 | CF(Cl)₂ | F | —CH₂—CF₃ | |
| 229 | CF(Cl)₂ | F | —CF₂—CHF₂ | |
| 230 | CF(Cl)₂ | F | —CF₂—CF₃ | |
| 231 | CF(Cl)₂ | F | —CF₂—CHF—Cl | |
| 232 | CF(Cl)₂ | F | —CF₂—CHF—CF₃ | |
| 233 | CF(Cl)₂ | F | —CH(CF₃)₂ | |
| 234 | CF(Cl)₂ | F | —CF₂—CF₂—CF₃ | |
| 235 | CF(Cl)₂ | F | —CF(CF₃)₂ | |
| 236 | CF(Cl)₂ | F | —CH₂—CF₂—Br | |
| 237 | CF(Cl)₂ | F | —CH₂—CF₂—CHF₂ | |
| 238 | CF(Cl)₂ | F | —CH₂—CF₂—CF₂—CF₃ | |
| 239 | CF(Cl)₂ | F | —CF₂—CF₂—CF₂—CF₃ | |
| 240 | CF(Cl)₂ | F | —CH₂—C(Cl)═CH₂ | |
| 241 | CHF—CH₃ | F | —CHF₂ | |
| 242 | CHF—CH₃ | F | —CF₃ | |
| 243 | CHF—CH₃ | F | —CH₂—CHF₂ | |
| 244 | CHF—CH₃ | F | —CH₂—CF₃ | |
| 245 | CHF—CH₃ | F | —CF₂—CHF₂ | |
| 246 | CHF—CH₃ | F | —CF₂—CF₃ | |
| 247 | CHF—CH₃ | F | —CF₂—CHF—Cl | |
| 248 | CHF—CH₃ | F | —CF₂—CHF—CF₃ | |
| 249 | CHF—CH₃ | F | —CH(CF₃)₂ | |
| 250 | CHF—CH₃ | F | —CF₂—CF₂—CF₃ | |
| 251 | CHF—CH₃ | F | —CF(CF₃)₂ | |
| 252 | CHF—CH₃ | F | —CH₂—CF₂—Br | |
| 253 | CHF—CH₃ | F | —CH₂—CF₂—CHF₂ | |
| 254 | CHF—CH₃ | F | —CH₂—CF₂—CF₂—CF₃ | |
| 255 | CHF—CH₃ | F | —CF₂—CF₂—CF₂—CF₃ | |

TABLE B
Compounds I in which Het is a
thiazole radical of the formula IIb:

| Com- | | | | |
|---|---|---|---|---|
| pound | | | | |
| No. | R³ | R⁴ | X | Physical data |
| 301 | CH₃ | CH₃ | —CHF₂ | |
| 302 | CH₃ | CH₃ | —CF₃ | |
| 303 | CH₃ | CH₃ | —CH₂—CHF₂ | |
| 304 | CH₃ | CH₃ | —CH₂—CF₃ | |
| 305 | CH₃ | CH₃ | —CF₂—CHF₂ | 134-135° C. |
| 306 | CH₃ | CH₃ | —CF₂—CF₃ | |
| 307 | CH₃ | CH₃ | —CF₂—CHF—Cl | |
| 308 | CH₃ | CH₃ | —CF₂—CHF—CF₃ | |
| 309 | CH₃ | CH₃ | —CH(CF₃)₂ | |
| 310 | CH₃ | CH₃ | —CF₂—CF₂—CF₃ | |
| 311 | CH₃ | CH₃ | —CF(CF₃)₂ | |
| 312 | CH₃ | CH₃ | —CH₂—CF₂—Br | |
| 313 | CH₃ | CH₃ | —CH₂—CF₂—CHF₂ | |
| 314 | CH₃ | CH₃ | —CH₂—CF₂—CF₂—CF₃ | |
| 315 | CH₃ | CH₃ | —CF₂—CF₂—CF₂—CF₃ | |
| 316 | CH₃ | CH₃ | —CH₂—C(Cl)═CH₂ | |
| 317 | CHF₂ | CH₃ | —CHF₂ | |
| 318 | CHF₂ | CH₃ | —CF₃ | |
| 319 | CHF₂ | CH₃ | —CH₂—CHF₂ | |
| 320 | CHF₂ | CH₃ | —CH₂—CF₃ | |
| 321 | CHF₂ | CH₃ | —CF₂—CHF₂ | 89-92° C. |
| 322 | CHF₂ | CH₃ | —CF₂—CF₃ | |
| 323 | CHF₂ | CH₃ | —CF₂—CHF—Cl | |
| 324 | CHF₂ | CH₃ | —CF₂—CHF—CF₃ | |
| 325 | CHF₂ | CH₃ | —CH(CF₃)₂ | |
| 326 | CHF₂ | CH₃ | —CF₂—CF₂—CF₃ | |
| 327 | CHF₂ | CH₃ | —CF(CF₃)₂ | |
| 328 | CHF₂ | CH₃ | —CH₂—CF₂—Br | |
| 329 | CHF₂ | CH₃ | —CH₂—CF₂—CHF₂ | |
| 330 | CHF₂ | CH₃ | —CH₂—CF₂—CF₂—CF₃ | |
| 331 | CHF₂ | CH₃ | —CF₂—CF₂—CF₂—CF₃ | |
| 332 | CHF₂ | CH₃ | —CH₂—C(Cl)═CH₂ | |
| 333 | CF₃ | CH₃ | —CHF₂ | |
| 334 | CF₃ | CH₃ | —CF₃ | |
| 335 | CF₃ | CH₃ | —CH₂—CHF₂ | |
| 336 | CF₃ | CH₃ | —CH₂—CF₃ | |
| 337 | CF₃ | CH₃ | —CF₂—CHF₂ | 118-120° C. |
| 338 | CF₃ | CH₃ | —CF₂—CF₃ | |
| 339 | CF₃ | CH₃ | —CF₂—CHF—Cl | 74-76° C. |
| 340 | CF₃ | CH₃ | —CF₂—CHF—CF₃ | 70-71° C. |
| 341 | CF₃ | CH₃ | —CH(CF₃)₂ | |
| 342 | CF₃ | CH₃ | —CF₂—CF₂—CF₃ | |
| 343 | CF₃ | CH₃ | —CF(CF₃)₂ | |
| 344 | CF₃ | CH₃ | —CH₂—CF₂—Br | 75-77° C. |
| 345 | CF₃ | CH₃ | —CH₂—CF₂—CHF₂ | |
| 346 | CF₃ | CH₃ | —CH₂—CF₂—CF₂—CF₃ | |
| 347 | CF₃ | CH₃ | —CF₂—CF₂—CF₂—CF₃ | |
| 348 | CF₃ | CH₃ | —CH₂—C(Cl)═CH₂ | |
| 349 | CHF—CH₃ | CH₃ | —CHF₂ | |
| 350 | CHF—CH₃ | CH₃ | —CF₃ | |
| 351 | CHF—CH₃ | CH₃ | —CH₂—CHF₂ | |
| 352 | CHF—CH₃ | CH₃ | —CH₂—CF₃ | |
| 353 | CHF—CH₃ | CH₃ | —CF₂—CHF₂ | 114-115° C. |
| 354 | CHF—CH₃ | CH₃ | —CF₂—CF₃ | |
| 355 | CHF—CH₃ | CH₃ | —CF₂—CHF—Cl | |
| 356 | CHF—CH₃ | CH₃ | —CF₂—CHF—CF₃ | |
| 357 | CHF—CH₃ | CH₃ | —CH(CF₃)₂ | |
| 358 | CHF—CH₃ | CH₃ | —CF₂—CF₂—CF₃ | |
| 359 | CHF—CH₃ | CH₃ | —CF(CF₃)₂ | |
| 360 | CHF—CH₃ | CH₃ | —CH₂—CF₂—Br | |
| 361 | CHF—CH₃ | CH₃ | —CH₂—CF₂—CHF₂ | |
| 362 | CHF—CH₃ | CH₃ | —CH₂—CF₂—CF₂—CF₃ | |
| 363 | CHF—CH₃ | CH₃ | —CF₂—CF₂—CF₂—CF₃ | |
| 364 | CHF—CH₃ | CH₃ | —CH₂—C(Cl)═CH₂ | |

TABLE C
Compounds I in which Het is a
pyridine radical of the formula IIc:

| Compound No. | R⁵ | X | Physical data |
|---|---|---|---|
| 401 | Cl | —CHF₂ | |
| 402 | Cl | —CF₃ | |
| 403 | Cl | —CH₂—CHF₂ | 123-125° C. |
| 404 | Cl | —CH₂—CF₃ | 137-138° C. |
| 405 | Cl | —CF₂—CHF₂ | 125-126° C. |
| 406 | Cl | —CF₂—CF₃ | |
| 407 | Cl | —CF₂—CHF—Cl | 73-76° C. |
| 408 | Cl | —CF₂—CHF—CF₃ | 72-75° C. |
| 409 | Cl | —CH(CF₃)₂ | |
| 410 | Cl | —CF₂—CF₂—CF₃ | |
| 411 | Cl | —CF(CF₃)₂ | |
| 412 | Cl | —CH₂—CF₂—Br | 55-60° C. |
| 413 | Cl | —CH₂—CF₂—CHF₂ | 107-109° C. |
| 414 | Cl | —CH₂—CF₂—CF₂—CF₃ | 131-133° C. |
| 415 | Cl | —CF₂—CF₂—CF₂—CF₃ | |
| 416 | Cl | —CH₂—C(Cl)═CH₂ | 63-65° C. |
| 417 | F | —CHF₂ | |
| 418 | F | —CF₃ | |
| 419 | F | —CH₂—CHF₂ | |
| 420 | F | —CH₂—CF₃ | |
| 421 | F | —CF₂—CHF₂ | |
| 422 | F | —CF₂—CF₃ | |
| 423 | F | —CF₂—CHF—Cl | |
| 424 | F | —CF₂—CHF—CF₃ | |
| 425 | F | —CH(CF₃)₂ | |
| 426 | F | —CF₂—CF₂—CF₃ | |
| 427 | F | —CF(CF₃)₂ | |
| 428 | F | —CH₂—CF₂—Br | |
| 429 | F | —CH₂—CF₂—CHF₂ | |
| 430 | F | —CH₂—CF₂—CF₂—CF₃ | |
| 431 | F | —CF₂—CF₂—CF₂—CF₃ | |
| 432 | F | —CH₂—C(Cl)═CH₂ | |
| 433 | CF₃ | —CHF₂ | |
| 434 | CF₃ | —CF₃ | |
| 435 | CF₃ | —CH₂—CHF₂ | |
| 436 | CF₃ | —CH₂—CF₃ | |
| 437 | CF₃ | —CF₂—CHF₂ | |
| 438 | CF₃ | —CF₂—CF₃ | |
| 439 | CF₃ | —CF₂—CHF—Cl | |
| 440 | CF₃ | —CF₂—CHF—CF₃ | |
| 441 | CF₃ | —CH(CF₃)₂ | |
| 442 | CF₃ | —CF₂—CF₂—CF₃ | |
| 443 | CF₃ | —CF(CF₃)₂ | |
| 444 | CF₃ | —CH₂—CF₂—Br | |
| 445 | CF₃ | —CH₂—CF₂—CHF₂ | |
| 446 | CF₃ | —CH₂—CF₂—CF₂—CF₃ | |
| 447 | CF₃ | —CF₂—CF₂—CF₂—CF₃ | |
| 448 | CF₃ | —CH₂—C(Cl)═CH₂ | |

A considerably enhanced activity in the method according to the invention is achieved by using a heterocyclylcarboxanilide I together with at least one active compound II from the following group, in synergistically effective amounts:
azoles, such as bromoconazole, cyproconazole, difenoconazole, epoxiconazole, fluquinconazole, flusilazole, flutriafol, hexaconazole, imazalil, metconazole, myclobutanil, penconazole, propiconazole, prochloraz, prothioconazole, tebuconazole, tetraconazole, triadimefon, triadimenol, triticonazole,
acylalanines, such as benalaxyl, metalaxyl, mefenoxam, ofurace, oxadixyl,
amine derivatives, such as guazatine,
anilinopyrimidines, such as pyrimethanil, mepanipyrim or cyprodinil,
dicarboximides, such as iprodione, procymidone, vinclozolin,
dithiocarbamates, such as mancozeb, metiram, thiram,
heterocylic compounds, such as benomyl, boscalid, carbendazim, carboxin, oxycarboxin, fuberidazole, picobenzamid, penthiopyrad, proquinazid, thiabendazole, thiophanate-methyl, dodemorph, fenpropimorph, tridemorph, 5-chloro-7-(4-methylpiperidin-1-yl)-6-(2,4,6-trifluorophenyl)-[1,2,4]triazolo[1,5-a]pyrimidine,
phenylpyrroles, such as fenpiclonil or fludioxonil,
other fungicides, such as benthiavalicarb, cyflufenamid, fosetyl, fosetyl-aluminum, phosphorous acid or its salts, iprovalicarb, metrafenone,
strobilurins, such as azoxystrobin, dimoxystrobin, enestrobin, enestroburin, fluoxastrobin, kresoxim-methyl, metominostrobin, orysastrobin, picoxystrobin, pyraclostrobin, trifloxystrobin, methyl (2-chloro-5-[1-(3-methylbenzyloxyimino)-ethyl]benzyl)carbamate, methyl (2-chloro-5-[1-(6-methylpyridin-2-ylmethoxy-imino)ethyl]benzyl)carbamate and methyl 2-ortho-[(2,5-dimethylphenyloxy-methylene)phenyl]-3-methoxyacrylate;
cinnamides and analogs, such as dimethomorph, flumetover or flumorph.

It has been found that simultaneous, that is joint or separate, application of a heterocyclylcarboxanilide I and at least one compound II or successive application of a heterocyclylcarboxanilide I and an active compound II allows better control of rust infections in leguminous plants than is possible with the individual compounds (synergistic mixtures).

Accordingly, the invention also relates to fungicidal mixtures for controlling rust fungi, which mixtures comprise, as active components,

A) a heterocyclylcarboxanilide I, and
B) an active compound II as defined above
in synergistically effective amounts.

The heterocyclylcarboxanilide I and the active compound II can be applied simultaneously, that is jointly or separately, or in succession, the sequence, in the case of separate application, generally not having any effect on the result of the control measures.

The harmful fungi are controlled by applying the heterocyclylcarboxanilides I by treating the seed, by spraying or dusting the plants or the soil before or after sowing of the plants, or before or after emergence of the plants.

The rust diseases in leguminous plants are controlled advantageously by applying an aqueous preparation of a formulation comprising a heterocyclylcarboxanilide I to the above-ground parts of the plants, in particular the leaves, or, as a prophylactic on account of the high systemic effectiveness, by treating the seed or the soil.

The compounds I and II are usually applied in a weight ratio of from 100:1 to 1:100, preferably from 20:1 to 1:20, in particular from 10:1 to 1:10.

In the method according to the invention, the heterocyclylcarboxanilide I can advantageously be applied together with other active compounds III, in addition to the active compounds II also with herbicides, insecticides, growth regulators, further fungicides or else with fertilizers. Suitable further mixing partners III of this nature are in particular:
imazethapyr, imazamox, imazapyr, imazapic or dimethenamid-p;
fipronil, carbofuran, carbosulfan, benfuracarb, MTI 446, CGA 293343 or a neonicotinoide insecticide such as imidacloprid, acetamipird, nitenpyram, thiacloprid, clothianidin, dinotefuran and thiamethoxam.

The compounds I and III are usually applied in a weight ratio of from 100:1 to 1:100, preferably from 20:1 to 1:20, in particular from 10:1 to 1:10.

The mixtures, described above, of a heterocyclylcarboxanilide I with herbicides are used in particular in crops in which the sensitivity of the plants to these herbicides, in particular the active imidazolinone compounds, is reduced.

When the heterocyclylcarboxanilides I are used in soybeans, the yields are increased considerably. Thus, the heterocyclylcarboxanilides I may also be used to increase the yield. By virtue of the yield increase in combination with the excellent action of heterocyclylcarboxanilides I against rust diseases in leguminous plants, the method according to the invention is of particular benefit to the farmer. Excellent results can be achieved by using a heterocyclylcarboxanilide I in combination with an active compound II.

The method according to the invention also allows very good control of other harmful fungi frequently encountered in leguminous plants. The most important fungal diseases in soybeans are the following:
Microsphaera diffusa
Cercospora kikuchi
Cercospora sojina
Septoria glycines
Colletotrichum truncatum

The heterocyclylcarboxanilides I and the mixtures, described above, of I and II are also suitable for controlling the abovementioned diseases.

The heterocyclylcarboxanilides I and the mixtures of I and II are applied by treating the fungi or the plants or materials to be protected against fungal attack or the soil with a fungicidally effective amount of the active compounds. Application can be both before and after the infection of the materials or plants with the fungi. The treatment is preferably carried out prior to the infection.

The fungicidal compositions generally comprise from 0.1 to 95% by weight, preferably from 0.5 to 90% by weight, of the active compound.

When a heterocyclylcarboxanilide I is used on its own, the application rates in the method according to the invention are from 0.01 to 1.5 kg of active compound per ha, depending on the type of effect desired.

In the treatment of seed, the amounts of active compound required are generally from 1 to 1500 g of heterocyclylcarboxanilide 1, preferably from 10 to 500 g, per 100 kilogram of seed.

Depending on the type of active compound II and the desired effect, the application rates of the mixtures according to the invention are from 10 g/ha to 2500 g/ha, preferably from 50 to 2000 g/ha, in particular from 100 to 1500 g/ha.

When using the mixtures, the application rates for heterocyclylcarboxanilide I are correspondingly generally from 1 to 1000 g/ha, preferably from 10 to 750 g/ha, in particular from 20 to 500 g/ha.

Correspondingly, the application rates for the active compound II are generally from 1 to 1500 g/ha, preferably from 10 to 1250 g/ha, in particular from 20 to 1000 g/ha.

In the treatment of seed, application rates of mixture are generally from 1 to 2000 g/100 kg of seed, preferably from 1 to 1500 g/100 kg, in particular from 5 to 1000 g/100 kg of seed.

For use in the method according to the invention, the compounds can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone, N-octylpyrrolidone), acetates (glycol diacetate), glycols, fatty acid dimethylamides, fatty acids and fatty acid esters. In (principle, solvent mixtures may also be used.
carriers such as ground natural minerals (for example kaolins, clays, talc, chalk) and ground synthetic minerals (for example highly disperse silicic acid, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR or HPLC spectrum).

The following are examples of formulations:

1. Products for Dilution with Water

### A) Water-Soluble Concentrates (SL)

10 parts by weight of a compound according to the invention are dissolved with 90 parts by weight of water or a water-soluble solvent. As an alternative, wetting agents or other auxiliaries are added. The active compound dissolves upon dilution with water. This gives a formulation having an active compound content of 10% by weight.

### B) Dispersible Concentrates (DC)

20 parts by weight of a compound according to the invention are dissolved in 70 parts by weight of cyclohexanone with addition of 10 parts by weight of a dispersant, for example polyvinylpyrrolidone. Dilution with water gives a dispersion. The active compound concentration is 20% by weight

### C) Emulsifiable Concentrates (EC)

15 parts by weight of a compound according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D) Emulsions (EW, EO)

25 parts by weight of a compound according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E) Suspensions (SC, OD)

In an agitated ball mill, 20 parts by weight of a compound according to the invention are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

### F) Water-Dispersible Granules and Water-Soluble Granules (WG, SG)

50 parts by weight of a compound according to the invention are ground finely with addition of 50 parts by weight of dispersants and wetting agents and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G) Water-Dispersible Powders and Water-Soluble Powders (WP, SP)

75 parts by weight of a compound according to the invention are ground in a rotor-stator mill with addition of 25 parts by weight of dispersants, wetting agents and silica gel. Dilution with water gives a stable dispersion or solution of the active compound. The active compound content of the formulation is 75% by weight.

### 2. Products to be Applied Undiluted

### H) Dustable Powders (DP)

5 parts by weight of a compound according to the invention are ground finely and mixed intimately with 95 parts by weight of finely divided kaolin. This gives a dustable product with an active compound content of 5% by weight.

### J) Granules (GR, FG, GG, MG)

0.5 part by weight of a compound according to the invention is ground finely and associated with 99.5 parts by weight of carriers. Current methods are extrusion, spray-drying or the fluidized bed. This gives granules with an active compound content of 0.5% by weight to be applied undiluted.

K) ULV solutions (UL)

10 parts by weight of a compound according to the invention are dissolved in 90 parts by weight of an organic solvent, for example xylene. This gives a product with an active compound content of 10% by weight to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. However, it is also possible to prepare concentrates composed of active substance, wetting agent, tackifier, dispersant or emulsifier and, if appropriate, solvent or oil, with these concentrates being suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even to apply the active compound without additives.

Oils of various types, wetting agents, adjuvants, herbicides, fungicides, other pesticides, or bactericides may be added to the active compounds even, if appropriate, not until immediately prior to use (tank mix). These agents are typically admixed with the compositions according to the invention in a weight ratio of from 1:100 to 100:1, preferably from 1:10 to 10:1.

### USE EXAMPLE

The active compounds were prepared as a stock solution comprising 25 mg of heterocyclylcarboxanilide 1, which was made up to 10 ml using a mixture of acetone and/or dimethyl sulfoxide and the emulsifier Uniperol® EL (wetting agent having an emulsifying and dispersing action based on ethoxylated alkylphenols) in a ratio by volume of solvent/emulsifier of 99:1. The mixture was then made up to 100 ml with water. This stock solution was diluted with the solvent/emulsifier/water mixture described to give the concentration of active compound stated below. Alternatively, the active compounds were employed as a commercial finished formulation and diluted with water to the stated concentration of active compound.

Curative Activity Against Soybean Rust Caused by Phakopsora pachyrhizi

Leaves of potted soybean seedlings were inoculated with a spore suspension of soybean rust (Phakopsora pachyrhizi). The pots were then placed into a chamber with high atmospheric humidity (90-95%) at 23 to 27° C. for 24 hours. After 2 days, the infected plants were sprayed to runoff point with the active compound solution described above at the concentration of active compound stated below. After the spray coating had dried on, the test plants were cultivated in a greenhouse at temperatures between 23 and 27° C. and at 60 to 80% relative atmospheric humidity for 14 days. The extent of the rust fungus development on the leaves was then determined.

After 2 days, the plants which had been treated with 63 ppm of the compound No. 21, 40, 53, 56, 69, 181, 339, 408 and 437 showed a rust infection of at most 26%, whereas the untreated plants were 70% infected.

### Seed Dressing Trial, Activity Against Soybean Rust

Soybean seeds of the cultivar BRS 133 were treated with 1000 g of a heterocyclylcarboxanilide I/100 kg of seed, formulated as SC with 250 g of active compound per liter, as liquid dressing, then sown into pots and cultivated in a greenhouse at about 22° C. 3 weeks after sowing, the plants were inoculated with soybean rust, incubated at 100% relative atmospheric humidity for 24 hours and then again cultivated in the greenhouse. At the time of inoculation, the first pair of leaves and one subsequent leaf had developed. The infection of the leaves was assessed 11 days after the inoculation.

We claim:

1. A method for controlling rust infections in leguminous plants, which method comprises treating the plants, the seeds or the soil by spraying or dusting with a fungicidally effective amount of a heterocyclylcarboxanilide of the formula I 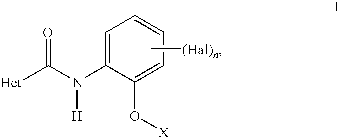
in which the variables are as defined below:
n is 0, 1, 2, 3 or 4;
Hal is halogen;
X is C₁-C₆-haloalkyl or C₂-C₆-haloalkenyl;
Het is a pyrazole, thiazole or pyridine radical of the formula IIa, IIb or IIc 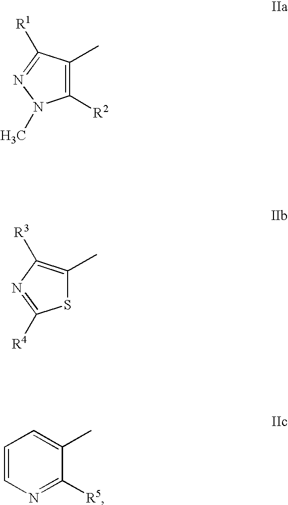
where
R¹ is C₁-C₄-alkyl or C₁-C₄-haloalkyl,
R² is hydrogen or halogen,
R³ is C₁-C₄-alkyl or C₁-C₄-haloalkyl,
R⁴ is C₁-C₄-alkyl or C₁-C₄-haloalkyl and
R⁵ is halogen, C₁-C₄-alkyl, C₁-C₄-haloalkyl, C₁-C₄-alkoxy, C₁-C₄-alkylthio, C₁-C₄-alkylsulfinyl or C₁-C₄-alkylsulfonyl.

2. The method according to claim 1, wherein in formula In is 0 and X is C₁-C₆-haloalkyl.

3. The method according to claim 1, wherein an aqueous preparation of a formulation comprising a heterocyclylcarboxanilide I is applied to the above-ground parts of the plants.

4. The method according to claim 1, wherein the rust infection is controlled by treating the seed or by treating the soil.

5. The method according to any of claims 1 to 4, wherein the harmful fungus Phakopsora pachyrhizi or Phakopsora meibomiae is controlled.

6. The method according to any of claims 1 to 4, wherein a combination of a heterocyclylcarboxanilide I with at least one commercial fungicide is employed.

7. The method according to any of claims 1 to 4, wherein a combination of a heterocyclylcarboxanilide I with at least one active compound II selected from the group below is used:
azoles, such as bromoconazole, cyproconazole, difenoconazole, epoxiconazole, fluquinconazole, flusilazole, flutriafol, hexaconazole, imazalil, metconazole, myclobutanil, penconazole, propiconazole, prochloraz, prothioconazole, tebuconazole, tetraconazole, triadimefon, triadimenol, triticonazole,
acylalanines, such as benalaxyl, metalaxyl, mefenoxam, ofurace, oxadixyl,
amine derivatives, such as guazatine,
anilinopyrimidines, such as pyrimethanil, mepanipyrim or cyprodinil,
dicarboximides, such as iprodione, procymidone, vinclozolin,
dithiocarbamates, such as mancozeb, metiram, thiram,
heterocylic compounds, such as benomyl, boscalid, carbendazim, carboxin, oxycarboxin, fuberidazole, picobenzamid, penthiopyrad, proquinazid, thiabendazole, thiophanate-methyl, dodemorph, fenpropimorph, tridemorph, 5-chloro-7-(4-methylpiperidin-1-yl)-6-(2,4,6-trifluorophenyl)-[1,2,4]triazolo[1,5-a]pyrimidine,
phenylpyrroles, such as fenpiclonil or fludioxonil,
other fungicides, such as benthiavalicarb, cyflufenamid, fosetyl, fosetyl-aluminum, phosphorous acid or its salts, iprovalicarb, metrafenone,
strobilurins, such as azoxystrobin, dimoxystrobin, enestrobin, enestroburin, fluoxastrobin, kresoxim-methyl, metominostrobin, orysastrobin, picoxystrobin, pyraclostrobin, trifloxystrobin, methyl (2-chloro-5-[1-(3-methylbenzyloxyimino)-ethyl]benzyl)carbamate, methyl (2-chloro-5-[1-(6-methylpyridin-2-ylmethoxy-imino)ethyl]benzyl)carbamate and methyl 2-ortho-[(2,5-dimethylphenyloxy-methylene)phenyl]-3-methoxyacrylate;
cinnamides and analogs, such as dimethomorph, flumetover or flumorph.

8. The method according to any of claims 1 to 4, wherein a combination of heterocyclylcarboxanilide I with at least one commercial herbicide tolerated by leguminous plants is employed.

9. The method according to any of claims 1 to 4, wherein a combination of heterocyclylcarboxanilide I with at least one commercial insecticide is employed.

10. The method according to any of claims 1 to 4, wherein a combination of heterocyclylcarboxanilide I with at least one active compound selected from the group below is used:
imazethapyr, imazamox, imazapyr, imazapic or dimethenamid-p;
fipronil, carbofuran, carbosulfan, benfuracarb, MTI 446, CGA 293343 or a neonicotinoide insecticide.

11. The method according to any of claims 6 to 10, wherein the heterocyclylcarboxanilide I and the second active compound are applied simultaneously, that is together or separately, or in succession.

12. The method according to any of claims 6 to 10, wherein the mixture according to to any of claims 6 to 10 is applied in an amount of from 5 g/ha to 2500 g/ha.

13. A fungicidal mixture for controlling rust fungi, which mixture comprises as active components
A) a heterocyclylcarboxanilide I according to claim 1, and
B) at least one active compound II according to claim 6 or 7
in synergistically effective amounts.

14. The fungicidal mixture according to claim 10, which comprises as active component II an azole according to claim 7.

15. The fungicidal mixture according to claim 13 or 14, which comprises a heterocyclylcarboxanilide I and the active compound II in a weight ratio of from 100:1 to 1:100.

16. A fungicidal mixture which comprises as active components
A) a heterocyclylcarboxanilide I according to claim 1, and
B) imazethapyr, imazamox, imazapyr, imazapic or dimethenamid-p
in a weight ratio of from 100:1 to 1:100.

17. A composition, comprising a liquid or solid carrier and a mixture according to any of claims 13 to 16.

18. The method according to any of claims 1 to 6, wherein a heterocyclylcarboxanilide I according to claim 1 or a mixture according to any of claims 6 to 10 is applied in an amount of from 1 to 2000 g/100 kg seed.

19. Seed, comprising the mixture according to any of claims 13 to 16 in an amount of from 1 to 2000 g/100 kg.

20. Use of at least one heterocyclylcarboxanilide I according to claim 1 and, if desired, a further active compound according to any of claims 6 to 10 for preparing a composition suitable for controlling rust infections in leguminous plants.
